(19) United States
(12) Patent Application Publication
(10) Pub. No.: US 2026/0314911 A1
(43) Pub. Date: Oct. 15, 2026

(54) RECORDING DEVICE FOR MEDICAL IMAGE DATA ACQUISITION

(71) Applicant: KARL STORZ SE & Co. KG, Tuttlingen (DE)

(72) Inventors: Simon Haag, Rietheim-Weilheim (DE); Jasmin Ilg, Dürbheim (DE)

(73) Assignee: KARL STORZ SE & Co. KG, Tuttlingen (DE)

(21) Appl. No.: 19/477,998

(22) PCT Filed: Apr. 23, 2024
(86) PCT No.: PCT/EP2024/061000

§ 371 Date: Oct. 23, 2025

(30) Foreign Application Priority Data
Apr. 24, 2023 (DE) 10 2023 110 422.5

(51) Int. Cl. A61B 1/00

(52) U.S. Cl. CPC A61B 1/00059; A61B 1/00016; A61B 1/00018

(57) ABSTRACT

The present invention relates to a recording device (10) for medical image data, comprising a connection unit (1) for selectively connecting an imaging device (2) for a medical treatment, in particular an endoscope, a data acquisition unit (3) for acquiring image and/or video data (3a) as well as device data (3b) from at least the imaging device (2) which is connected to the connection unit (1), a documentation unit (5) for recording image and/or video data (3a) and device data (3b) provided by the data acquisition unit (3), wherein the device comprises a control unit (4) which is designed to determine and/or assign at least one attribute, preferably a plurality of attributes, for the imaging device (2) which is connected to the connection unit, and to control the documentation unit (5) to record depending on one or multiple assigned attribute(s).

The present invention relates to a device and a system for data acquisition and data transmission for medical data.

Optical visualization systems such as endoscopes or microscopes for medical procedures are well known and enable the representation of a scene and/or a work region within a human body, for example during a minimally invasive procedure. Such visualization systems output captured image or video data on suitable screens and are usually connected to an image recording device or an image recording system in order to record the captured image or video data as well as, if necessary, further devices and/or patient-specific data.

In the known image recording systems, the image or video data, as well as data relating to the devices/instruments used, are completely recorded by so-called data loggers in medical practices or hospitals and transferred to a memory device. These data can then be processed later, for example as historical data for monitoring the course of treatment or for analysis purposes. Furthermore, AI applications can also be trained using these data. For this purpose, the acquired data are usually manually reviewed, searched through for specific events or instruments in the image, and commented on accordingly. However, this is very time-consuming and also requires high data transfer volumes and computing power.

U.S. Pat. No. 7,738,940 B2 discloses a medical image recording system which, in addition to the image information generated by an endoscope, also records device information and patient information for a medical examination and can transmit this information to a data storage unit. The system comprises at least one image information acquisition unit and a device information acquisition unit that acquires information about a device used at the time of examination, and a recording unit. The system is designed to carry out a data storage process after completion of an examination, in which the patient data, device data and image data initially stored in a non-volatile memory of an image recording device are transferred via a network to a server of a database service.

WO 2018/013666 A1 discloses a system for managing medical devices that communicate with a local network. A local network application forwards the device data collected by the medical devices to a cloud-based service that processes and organizes the device data. The cloud-based service can also communicate with a sales database, an ERP system, a manufacturing database, and/or other databases. Users can request device data from the cloud-based service using a conventional web browser. The device data may include location data, usage data, repair data, etc.

Pablo Azarge et al., “EndoMapper dataset of complete calibrated endoscopy procedures,” describes the creation and provision of a dataset for training spatial artificial intelligence in endoscopy, in particular for developing a 3D mapping and localization system for detecting blind spots during the examination, automatically measuring polyps, and guiding the physician to a polyp. In this case, in addition to complete endoscopy sequences which were captured during regular medical practice, associated metadata, annotations from a treating physician, as well as anatomical landmarks and segmentation masks are captured and provided in the data set.

Based on the known prior art, it is the object of the present invention to provide a device and a system for data acquisition for medical data, which enables optimized data acquisition and data transmission and preferably optimized processing of these data, for example for AI applications.

This object is achieved by the data device and the system for data transmission according to the independent claims. The dependent claims describe advantageous developments of the present invention.

In a first aspect, the present invention relates to a recording device for medical image data, comprising a connection unit for selectively connecting an imaging device for a medical treatment, in particular an endoscope or exoscope, a data acquisition unit for acquiring image and/or video data as well as device data from at least the imaging device connected to the connection unit, and a documentation unit for recording image and/or video data and device data provided by the data acquisition unit, wherein the device has a control unit which is designed to determine and/or assign at least one, preferably a plurality of, attributes for the imaging device connected to the connection unit and to control the documentation unit to record depending on one or more assigned attributes.

The present invention enables the determination and/or assignment of attributes for or to a respective imaging device, which is connected to the device by means of the connection unit, with the aid of the control unit according to the invention. In this case, the determination and/or assignment of the at least one attribute for the respectively connected imaging device is preferably carried out automatically by the control unit according to the invention. In this case, different types of devices, in particular different endoscopes, can be recognized and/or determined and attributes assigned to them. The documentation unit is then controlled based on the determined and/or assigned attributes. In contrast to the prior art, this allows for selective and/or respective device-specific control of the documentation unit for optimized data recording and/or data transmission. In particular, this can significantly reduce the data acquisition effort required to provide suitable documentation data, and optimize the quality of the documentation data for a particular medical treatment. This is true in particular because the data logging devices known from the prior art generate a huge data load due to the large number of examinations in a hospital or in medical practice, which requires both high computing power and memory capacity. Likewise, the present invention enables a significantly simplified evaluation of the recorded data for AI applications compared to the prior art, since image and/or video data can be collected that are in particular tailored to the respective imaging device and/or provided with respectively associated attributes, which also enable optimized further processing during later data utilization, in particular for training AI applications.

The documentation unit for recording image and/or video data as well as device data provided by the data acquisition unit is preferably designed to link the respective image and/or video data with the device data acquired during a respective examination and to store them as mutually assigned data or as a correspondingly linked data set. Likewise, the respectively determined and/or assigned attributes for the respective imaging device can advantageously be saved as associated data or as an associated data set.

The documentation unit is advantageously designed to store the device data and/or the respectively assigned attributes as metadata of the respective image and/or video data. This allows the image and/or video data to be clearly assigned to the device data, also at a later point in time. In particular, influences of human error can be avoided in the separate recording of image and/or video data known from the prior art on the one hand, and in the respective examination of additionally recorded data sets on the other hand. In a preferred embodiment, the documentation unit is designed to store the respective device data as metadata of the image and/or video data in predefined frames or, more preferably, in each individual frame of the image and/or video data.

The control unit is advantageously designed to determine and/or assign the respective attributes based on device data provided by the data acquisition unit. The data acquisition unit can, for example, be designed here to retrieve a device identification number or the like from the image acquisition device connected to the connection unit by means of suitable electronic data acquisition, which device identification number identifies the connected, specific imaging device. Based on this, associated attributes can then be determined, for example from an internally or externally stored table with corresponding attributes and/or from externally retrievable information relating to the respective device type.

The control unit is further advantageously designed to determine at least one type of medical treatment based on the data provided by the data acquisition unit and/or the imaging device as an attribute. By way of example, the control unit can determine the respective field of study and/or type of intervention, and thus the medical treatment, based on recognition of the connected endoscope. It is also possible to determine the expected image and/or video data. For example, if a flexible rhinoscope is connected to the device, then the examination performed by the physician is a rhino-laryngoscopy. This can be determined by the control unit as an attribute for the type of medical treatment and used for further data recording and/or data processing. Furthermore, the control unit can determine in this case that the connected endoscope does not have a working channel and is therefore not used in connection with high-frequency applications, for example in high-frequency or laser surgery. This can also be determined as a corresponding attribute of the connected endoscope and used for further data recording and/or data processing. Due to the unsuitability of the connected endoscope for high-frequency applications, it can further be determined or inferred that no smoke development will be visible in the image and/or video data during the treatment performed. This can also be determined as another attribute of the connected endoscope and used for further data recording and/or data processing. This can, for example, simplify the training of AI applications for smoke gas detection during treatment, since the determined attributes can be used to directly determine whether the collected data is suitable for training the AI application. Furthermore, for example, the image or video data can only be captured or activated by the control unit if an attribute of the image and/or video data required for a desired AI training is present or has been determined.

In a preferred embodiment, the attributes comprise one or more data items or data sets relating to one or more of the following properties: type of imaging device, medical specialty, medical indication, application medium, intended use, type of medical procedure, high-frequency application, laser application, instrument visibility, smoke development, bleeding, visual obstruction, and/or contaminated water.

The type of holding system used for the imaging device, such as a robot, can also be an attribute.

In a preferred embodiment, the control unit is designed to detect a data command or data acquisition command transmitted by the device and/or to the device and to use it for the attribute determination and/or assignment, and/or for the control of the documentation unit. In this way, for example, a data acquisition command, in particular one supplied externally, can be received by the device, which can then influence the further data acquisition by means of the device. For example, the control unit can be designed to compare the recorded data acquisition command with at least one attribute assignment of a connected endoscope and to control the documentation unit based thereon. In this case, for example a data acquisition command can be recorded which, in subsequent examinations, only requests data acquisition for training purposes for a specific AI application, such as detecting smoke development. Based on such a data acquisition command, the control unit can only activate the documentation unit if a corresponding attribute is recognized and/or assigned for the respectively connected image acquisition device. This allows for targeted data collection, which reduces computing power and computing capacity while simultaneously enabling targeted data transmission. This is a significant improvement compared with the prior art, where either all data are transmitted, which results in a huge amount of data transfer and data storage effort, or a respective complex manual pre-setting has to be carried out, which is undesirable for a treating physician.

In a preferred embodiment, the control unit is designed to control the documentation unit to activate and/or deactivate the recording, and/or to set and/or adapt recording parameters influencing the data recording, depending on the attributes. In particular, in this case, a selective activation and/or deactivation of the data recording or the documentation unit can take place, for example, when a predefined attribute has been recognized or assigned. For example, a specific video sequence can be saved based on one or more recognized attributes. Furthermore, for example, a setting or adjustment of a frame rate, an image section and/or other image parameters of the captured image and/or video signal can be carried out depending on the determined and/or assigned attributes.

The control device advantageously comprises a memory unit which is designed to store and/or assign a plurality of attributes for a respective imaging device. The storage unit may in particular comprise one or more lookup tables by means of which predefined and/or changeable attributes are assigned to a respective imaging device. Furthermore, the control device can be designed to determine attributes from the captured image and/or video data. Further preferably, the control device can be designed to determine attributes of the respective imaging device from a combination of the acquired device data and the acquired image and/or video data.

In a preferred embodiment, the data acquisition unit is designed to acquire further device data and/or parameter data from devices connected to the device in a wired or wireless manner. The data acquisition unit is further advantageously designed to acquire provided patient data and/or vital parameters of a patient. In this case, the control device can advantageously be designed to determine attributes of the respective image acquisition device or the respective medical intervention from a combination of the acquired device data and/or parameter data as well as the acquired image and/or video data.

In this way, information for example about an indication for the respective procedure can also be determined using the device data. For example, the data provided by an insufflator can be used to determine whether the person being treated is an adult or a small child. In this case, a pressure value of the insufflator can also provide information about the patient's weight, since an obese patient requires a higher pressure than a normal-weight patient. The data from a suction irrigation pump can provide information about the specialty of the respective examination, as it has different modes, including in particular a “Gyn,” “Arthro,” and “Uro” mode, i.e., modes that are assigned to specific medical specialties.

In a preferred embodiment, the recording device further comprises a data transmission unit which is designed for preferably bidirectional data exchange with an external network and/or an external server unit. In this case, the data transmission unit can have a LAN and/or WLAN interface for connection to an internal or external network, such as a hospital LAN or the Internet. In this case, the recording device can be designed to acquire data acquisition commands from the external server unit and/or to transmit recordings from the documentation unit to the external server unit in an automated and/or non-automated manner. The transmission to an external network and/or to an external server unit can take place in real time and/or with a time delay with respect to the recording of the documentation unit. Particularly preferably, the acquired data sets can be transmitted in each case at predefined time intervals or at predefined transmission times. In this case, the data can be temporarily stored in external or internal memory units.

The determined and/or assigned attributes are thus used in the form of a filter, in order to save only certain image or video data and, for example, to transmit said data for further use. The attributes can be logically dependent on one another or build on one another hierarchically, so that determining and/or assigning certain attributes ever further restricts the selection of other possible attributes. A set of attributes can thus describe and define a medical procedure or various states of the procedure or various events during the procedure as precisely as possible, in order to precisely control the documentation unit.

Likewise, an attribute newly determined or assigned during an intervention can serve to confirm previously determined attributes, so that a higher level of security in the control of the documentation unit is achieved in the form of a feedback loop. For example, an HF application can be suspected and assigned as an attribute based on the type of specialty and the equipment used, and then confirmed by smoke gas detected in the video image or information about an activated HF generator. It is possible to determine precise information such as a phase of the intervention, for example from the time of activation and deactivation of connected devices as an attribute.

The control of the documentation unit for saving image and video sequences can also be done depending on a determined relevance, for example the control could be done depending on the number or type of the determined and/or assigned attributes. Alternatively, the documentation unit can also be controlled depending on the absence of attributes.

In order to prevent a sudden start and end of a recording via the documentation unit, a time lead or lag can be provided when saving and/or transmitting image and video data by the documentation unit depending on the attributes. For example, it can be provided that the saved sequences have a certain minimum length of images.

The recording device described above can be designed as a separate or integral component of a medical imaging device, for example an endoscopy device, or of a medical imaging system, for example an endoscopy system.

In a further aspect, the present invention relates to a recording system comprising at least one recording device as described above, and at least one imaging device that can be selectively connected to a connection unit of the recording system, in particular an endoscope, preferably a plurality of different imaging devices, for capturing image and/or video data during a medical treatment.

The recording system advantageously comprises further devices which can be selectively connected to the recording system and which are designed to transmit device data and/or parameter data to a control unit of the recording device. These devices may comprise, for example, an insufflator and/or a suction irrigation pump.

In a preferred embodiment, the system further comprises a server unit which is connected to the recording device by means of an internal or external network and which is designed for preferably bidirectional data exchange with the recording device. In this case, the server unit can preferably be designed to transmit a data acquisition command to the at least one recording device, preferably to a plurality of recording devices of the system. In this case, the respective data acquisition command can contain attributes which can be used in the respective recording devices for targeted data acquisition during the respective medical treatments.

To avoid repetition, reference is made here to the above-mentioned statements regarding the recording device according to the invention, which are to be considered equally as disclosed and claimable for the system according to the invention.

Particular features, advantageous effects and details of the present invention are explained below with reference to the schematic drawings, merely by way of example.

In the drawings:

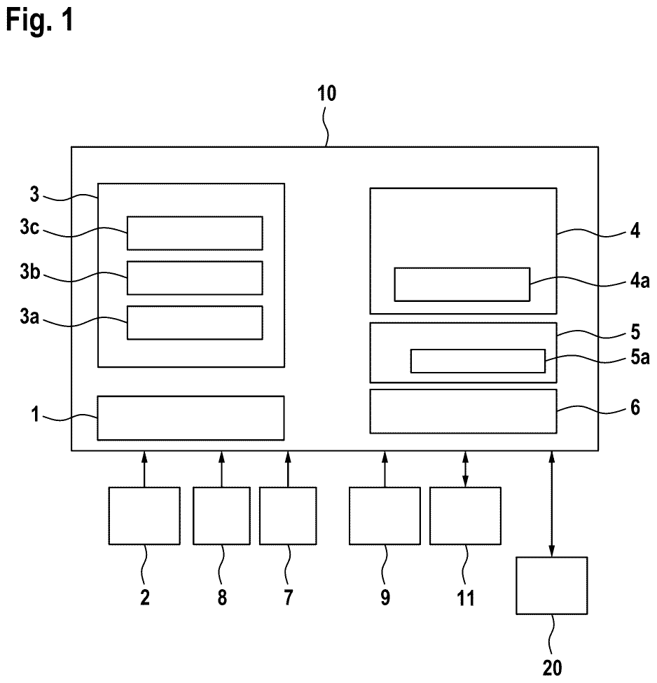

FIG. 1: shows a schematic representation of a preferred embodiment of the recording device for medical image data according to the invention; and

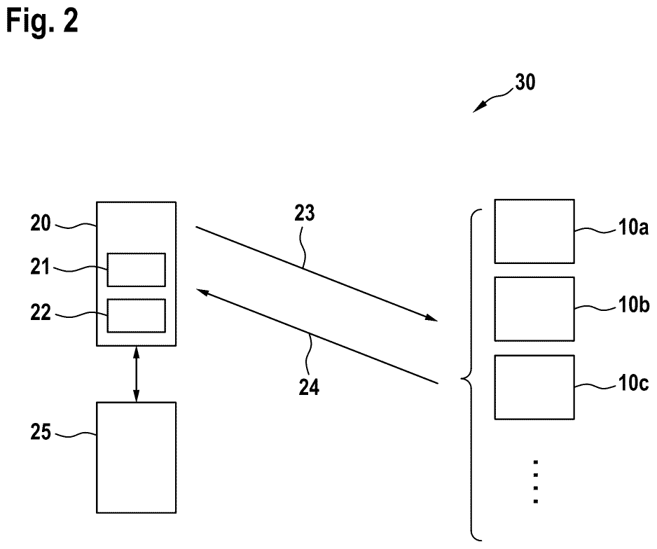

FIG. 2: shows a schematic representation of a preferred embodiment of a recording system according to the invention with a plurality of recording devices and an AI training unit connected to a server unit.

FIG. 1 shows a preferred embodiment of the recording device 10 for medical image data according to the invention. The device 10 initially comprises a connection unit 1 for selectively connecting an imaging device 2 for a medical treatment or a medical intervention. This is in particular an endoscope, more preferably a video endoscope. In this case, the connection unit can have plug connections which are known per se and which serve for the wired connection of the endoscope to the device.

The connection unit 1 can also be designed to connect further devices, such as an input unit 9, for example a keyboard and/or a mouse, a display unit 11 for outputting a captured video image to a treating physician, or additional devices such as an insufflator 7, a suction irrigation pump 8, etc. For this purpose, the connection unit 1 can have wired or wireless interfaces for the respective connection and/or communication with the devices 7, 8, 9, 11.

The device further comprises a data acquisition unit 3 for acquiring image and/or video data 3a provided by the imaging device 2. Furthermore, the data acquisition unit 3 is designed to acquire device data 3b from at least the imaging device 2 connected to the connection unit 1. In this case, the device data 3b can, for example, comprise a device identification number, a device identifier or the like, based on which an identification and/or recognition of the endoscope 2 connected to the device 10 is possible. Furthermore, the device data may comprise operating parameters or other information signals of the endoscope 2.

The data acquisition unit 3 can further be designed to acquire device data 3b from the optionally additionally connected devices 7, 8, 9, 11, in particular from a connected insufflator 7 and/or a suction irrigation pump 8. In this case, the acquired device data 3b can comprise, in particular, operating parameters of the devices, which can be used for the control and recording of the data described in more detail below.

The data acquisition unit 3 can additionally acquire patient data and/or vital parameters 3c associated with the patient, which can also be used for the control and recording described in more detail below. In this case, the patient data and/or vital parameters 3c can either be entered manually using appropriate input means 9 or transmitted automatically by the further devices 7, 8.

The device 10 further comprises a documentation unit 5 for recording image and/or video data 3a and device data 3b provided by the data acquisition unit 3. Furthermore, the documentation unit 5 can be designed to record patient data and/or vital parameters 3c provided by the data acquisition unit 3. For this purpose, the documentation unit 5 advantageously comprises an internal or external memory unit 5a, in particular a non-volatile memory, on which the supplied data can be saved.

Furthermore, the device 10 comprises a control unit 4, which is designed to determine and/or assign at least one, preferably a plurality of, attributes for the imaging device 2 connected to the connection unit 1 and to control the documentation unit 5 for recording depending on one or more assigned attributes.

Attributes are understood here as associated properties of the imaging device 2, in particular of the endoscope 2. For example, an attribute of the respective endoscope can be the type of medical treatment for which the endoscope is typically used. The attributes may further comprise, by way of example, data or data sets relating to the following properties: endoscope type, specialty, indication, medium used, intended use, high-frequency application, laser, instrument visibility, smoke development, and/or contaminated water. In a preferred embodiment, the attributes can be saved in the memory unit 4a of the control unit 4. In this case, the memory unit 4a can, for example, comprise tables or lookup tables in which an assignment of predefined attributes to individual imaging devices or endoscopes is predefined. The respective attributes can be determined by the control unit upon recognition of the respective endoscope based on the data provided by the data acquisition unit. A table by way of example with attributes and associated data of individual endoscope types is shown below.

TABLE 1

| | Attribute | | | | | | | | |
|---|---|---|---|---|---|---|---|---|---|
| | | | | | | Instruments | | Contaminated | Bladder |
| Endoscope | Specialty | Indication | Medium | HF | Laser | visible | Smoke | water | tumor |
| HD rhinoscope | ENT | Rhinolaryngoscopy | Air | No | No | No | No | No | no |
| HD view | Uro | Cystoscopy | Water | Yes | No | Yes | No | Yes | Yes |
| Tipcam | Lap | Cholecystectomy, | Air | Yes | No | Yes | Yes | No | no |
| | | inguinal hernia, | | | | | | | |
| | | appendectomy | | | | | | | |
| . . . | | | | | | | | | |

As can be seen from the table, some attributes such as “HF,” “Laser,” “Smoke,” “Instruments visible” can have a respective positive or negative value for an individual endoscope, depending on whether, for example, smoke development or the occurrence of contaminated water is possible or not with the endoscope used. Likewise, a respective attribute such as an indication or a specialty can have a more detailed, descriptive data set.

Furthermore, the control unit can be designed to assign attributes of a respectively connected endoscope based on the provided data. This may be the case, for example, if the control unit determines, based on the data stored in the memory unit 4a, that no attribute such as “medium” is stored for the recognized endoscope. In this case, the control unit can be designed to determine a respective attribute based on the acquired image and/or video data as well as the acquired device data. In this case, the control unit 4 can also be designed to access external data collections, external server units and/or external networks.

The control unit 4 is further advantageously designed to control the documentation unit 5 for activating and/or deactivating a recording, and/or for setting and/or adapting recording parameters, influencing a data recording, depending on the determined and/or assigned attributes. As a result, in contrast to pure data logging of all data during an examination with the endoscope, a recording can, for example, only be activated or deactivated if one or more predefined attributes are met or not met. This can be advantageous in particular for collecting data to train AI applications, as, for example, only data that appear to be useful for the respectively desired application are recorded. In addition or alternatively to the aforementioned embodiment, the control unit 4 can be configured to enable manual activation of the documentation unit 5, in particular by a treating physician. For example, data recording could be triggered in particular when there is for example interest in a particular form of treatment, so that the next time a similar procedure is available, the corresponding image and/or video data would also be saved.

If, for example, examination data and in particular video data with smoke gas in the image are required for a planned training of AI applications, the data of a particular examination can only be recorded if the determined and/or assigned attributes of the respectively connected endoscope indicate that smoke gas can be generated at all.

For example, if the physician uses a flexible rhinoscope, the control unit can determine that the medical treatment is a diagnostic rhinolaryngoscopy. In this case, no smoke will appear in the captured image or in the captured image and/or video data, as this endoscope does not have a working channel and therefore cannot be used in conjunction with HF instruments. However, if a flexible cystoscope is connected to the device, then it is a flexible cystoscopy. Therefore, it is likely that bladder tumors, mucosal redness and ostia will be visible in these videos. The information about the respectively connected endoscope or the respectively connected endoscope type can be used to determine what image content can be expected. Based on the respectively connected endoscope, unique attributes can thus be defined by the device, which serve for optimized data collection and transmission.

If, for example, image and video data from various instruments are needed to train an AI application, such as instrument recognition software, ideally only data sets that occur during an examination or procedure with a video cystoscope or another endoscope with a working channel should be collected. When using an HD rhinoscope, the data do not need to or should not be acquired and recorded for later training purposes, as this endoscope does not have a working channel and therefore cannot be used in combination with an instrument.

The control unit 4 can further be designed to evaluate device data 3b and in particular operating parameters of further devices 7, 8 connected to the recording device 10, such as an insufflator 7 or a suction irrigation pump 8, and to use them for determining and/or assigning attributes to a respectively connected imaging device 2. Thus, based on such device data or operating parameters, conclusions can be drawn, for example, about an indication for the respective examination. Thus, for example, an insufflation mode can be used to distinguish between an adult and a small child. The pressure of the insufflator can also be read out as an operating parameter. In particular, this can be used to determine the patient's weight.

Likewise, the control unit can be designed to combine the attribute assignment with the use of other AI applications. For example, when using an AI application for smoke detection, image and/or video data of a specific indication in which HF instruments are used can be collected. HF instruments sometimes look like normal instruments and can only be distinguished by their characteristics. However, the “HF” characteristic cannot be recognized in the image, only the “smoke” effect. Thus, the detection of the instruments can be linked to the formation of smoke. If both parameters are given, the control unit can assign the attribute HF instrument to the instrument.

Furthermore, the control unit can be designed to activate existing software functions or AI software functions, such as smoke gas detection, in a video image in the device only when they are actually needed. This allows computing power to be optimized. The control unit and/or the documentation unit can also be designed to automatically pre-sort collected image data based on the events expected from the respective instruments.

The device 10 further preferably comprises a data transmission unit 6, which is designed for preferably bidirectional data exchange with an external network and/or an external server unit 20. In this case, the data transmission unit 6 can have a LAN and/or WLAN interface for connection to an internal or external network, such as a hospital LAN or the Internet. This allows the device to transmit the recorded data to an external network and/or an external server unit 20. In this case, the respective transmission can take place during the actual recording of data and thus during medical treatment. Alternatively, each transfer can also take place at predefined intervals.

The recording device 10 can further be designed to acquire data acquisition commands by the external server unit 20 and/or to transmit recordings of the documentation unit 5 to the external server unit 20 in an automated and/or non-automated manner.

FIG. 2 is a schematic representation of a preferred embodiment of a recording system 30 according to the invention with a plurality of recording devices 10a, 10b, 10c and an AI training unit 25 connected to a server unit 20.

In this case, the server unit 20 is configured to communicate with a plurality of recording devices 10 via a suitable network such as an internal hospital network and/or the Internet. Here, the server unit 20 can issue a desired data acquisition command 23, which is transmitted to the individual recording devices 10a, 10b, 10c. In this case, the data acquisition command 23 advantageously comprises an instruction as to which attributes are of interest for further data evaluation or data utilization for an image and/or video recording during a respective medical procedure and therefore to be recorded by the individual recording devices 10a, 10b, 10c. The respective control unit of the recording devices 10a, 10b, 10c can then compare the acquired data acquisition command 23 with at least one attribute assignment of a connected endoscope 2 and, based thereon, control the documentation unit 5.

For example, the server unit 20 can issue a data acquisition command 23 which, in the subsequent examinations, only requests data acquisition for training purposes for a specific AI application, such as for detecting smoke development. In response to the desired data acquisition command, the respective control unit 4 can only activate the documentation unit if a corresponding attribute is recognized and/or assigned for the respectively connected imaging device. This allows for targeted data collection, which reduces computing power and computing capacity while simultaneously enabling targeted data transmission. The acquired data can be transmitted to the server unit 20 as documentation data 24. Furthermore, the respective control unit 4 can be designed, in response to the receipt of a corresponding data acquisition command 23, to search through previous recordings stored in the device 10a, 10b, 10c with regard to corresponding attributes and then to transmit them as documentation data 24 to the server unit 20.

The respective documentation data 24 can be acquired and stored in an internal or external data memory unit 21 of the server unit 20.

The server unit 20 may further comprise a computer unit 22 which can analyze, catalog, and/or prepare, for example segment, the documentation data 24 for training AI applications.

The server unit 20 may comprise or be connected to an internal or external AI training unit 25, which is designed to train AI software applications. The AI training unit 25 is advantageously designed to make the provided documentation data 24 available in a manner known per se for training an AI software application and/or a neural network.

The server unit 20 may further be configured to transmit an AI software application to the individual recording devices 10a, 10b, 10c connected thereto via the internal or external network.

### LIST OF REFERENCE SIGNS

1 connection unit
2 imaging device
3 data acquisition unit
3a image and/or video data
3b device data
3c patient data
4 control unit
4a memory unit
5 documentation unit
5a memory unit
6 data transmission unit
7.8 further devices
9 input means
10 recording device
11 display unit
20 server unit
21 data storage unit
22 computing unit
23 data storage commands
24 documentation data
25 AI training unit
30 recording system

1-17. (canceled)

18. A recording device for medical image data, comprising:
a connection unit;
a data acquisition unit;
a documentation unit;
a control unit; and
wherein the connection unit selectively connects to an imaging device;
wherein the data acquisition unit acquires at least one of image data, video data, and device data from the imaging device via the connection unit; and
wherein the documentation unit records the at least one of image data, video data, and device data provided by the data acquisition unit; and
wherein the control unit performs at least one of determining and assigning at least one attribute for the imaging device connected to the connection unit, and causing the documentation unit to record in accordance the at least one attribute.

19. The recording device of claim 1, wherein the control unit determines and/or assign the attributes based on device and/or image and/or video data provided by the data acquisition unit.

20. The recording device of claim 1, wherein the control unit determines at least one type of medical treatment as an attribute based on the recording device data and/or image and/or video data provided by the data acquisition unit.

21. The recording device of claim 1, wherein the control unit detects a data acquisition command transmitted by the recording device and/or to the recording device, and to use it for the attribute determination and/or assignment, and/or for the control of the documentation unit.

22. Recording device according to claim 21, wherein the control unit compares the detected data acquisition command with at least one attribute assignment and to control the documentation unit based thereon.

23. The recording device of claim 1, wherein the control unit controls the documentation unit for activating and/or deactivating a recording, and/or for setting and/or adapting recording parameters influencing a data recording depending on the attributes.

24. The recording device of claim 1, wherein the control device comprises a memory unit which is designed to store and/or assign a plurality of attributes for a respective imaging device.

25. The recording device of claim 1, wherein the data acquisition unit acquires further device data and/or parameter data from devices connected to the recording device in a wired or wireless manner.

26. The recording device of claim 1, wherein the data acquisition unit acquires provided patient data and/or vital parameters of a patient.

27. The recording device of claim 1, wherein the at least one attribute comprise one or more data items or data sets relating to the following properties: type of imaging device, medical specialty, medical indication, application medium, intended use, type of medical intervention, high-frequency application, laser application, instrument visibility, smoke development, bleeding, visual obstruction, and/or contaminated water.

28. The recording device of claim 1, wherein the recording device comprises a data transmission unit which is designed for preferably bidirectional data exchange with an external network and/or an external server unit.

29. The recording device of claim 28, wherein the recording device records data acquisition commands by the external server unit and/or to automatically and/or non-automatically transmit recordings of the documentation unit to the external server unit.

30. The recording device of claim 28, wherein the recording device transmits the recordings of the documentation unit in real time and/or with a time delay with respect to the recording, in particular at predefined time intervals.

31. The recording device of claim 1, wherein the data acquisition unit and/or the documentation unit is designed to store the respective device data as metadata of the image and/or video data and in particular as metadata in each individual frame of the image and/or video data.

32. The recording device of claim 1, wherein the imaging device is selectively connected to a recording system connection unit of the recording system to capturing image and/or video data during a medical treatment.

33. The recording device of claim 32, further comprising a number of further devices selectively connected to the recording system and which transmit device data and/or parameter data to a recording device control unit of the recording device.

34. The recording device of claim 32, further comprising a server connected to the recording device via at least one of an internal network or external network providing bidirectional data exchange with the recording device.
